(19) United States
(12) Patent Application Publication
(10) Pub. No.: US 2012/0201760 A1
(43) Pub. Date: Aug. 9, 2012

(54) Metal oxide particles coated with polyethylene glycol and their synthesis

(76) Inventors: Ulrich Ingmar Tromsdorf, Plankstadt (DE); Oliver Thomas Bruns, Hamburg (DE); Horst Weller, Hamburg (DE)

(21) Appl. No.: 13/389,160

(22) PCT Filed: Aug. 9, 2010
(86) PCT No.: PCT/EP2010/061547

§ 371 Date: Apr. 20, 2012

(30) Foreign Application Priority Data
Aug. 7, 2009 (GB) 0913803.3

(51) Int. Cl. A61K 49/18; A61P 43/00; A61K 49/00; A61K 9/14; B82Y 5/00; B82Y 40/00

(52) U.S. Cl. 424/9.322; 424/9.1; 424/490; 977/892; 977/773; 977/811; 977/927; 977/930; 977/915

(57) ABSTRACT

A T₁ blood pool contrast agent comprising very small iron oxide nanoparticles are coated with poly(ethylene glycol) (PEG) based ligands. Core size and length of the PEG chain were optimized according to stability, relaxometric properties, cytotoxicity and unspecified cell uptake.

### CROSS RELATION TO OTHER APPLICATIONS

This application claims the benefit of and priority to UK Patent Application No 0913803.3 filed on 7 Aug. 2009.

### FIELD OF THE INVENTION

The field of the invention relates to coated iron oxide nanoparticles having a diameter of less than 6 nm, their synthesis and use as T1 contrast agents in magnetic resonance imaging.

### BACKGROUND OF THE INVENTION

Superparamagnetic nanoparticles are of interest for various applications in biotechnology and biomedicine. Their unique magnetic properties can be fine tuned on the nanometre scale and make them particular promising in both diagnosis and therapy. Currently, one of the most important and rapidly growing fields is the use of iron oxide particles as contrast agents for Magnetic Resonance Imaging (MRI) (see, for example, Bjornerud et al, NMR in Biomedicine 2004; Bulte et al, NMR in Biomedicine 2004; Wang et al, European Radiology 2001, and Na et al, J. Mater Chem, 2009). MRI enables the visualisation of contrasts in images via monitoring of the response of water protons in soft tissues to external magnetic fields.

There is little difference in relaxation time between normal and abnormal soft tissues which leads to the resulting contrast the images. Thus additional supplements are used to enhance detection and diagnosis of the abnormal soft tissues. The most effective supplement is a so-called contrast agent which is introduced to a living body. The main task of the contrast agent in MRI is a shortening of the relaxation times T₁ and T₂ which characterize the two independent processes of proton relaxation of the water protons. One of the processes of proton relaxation is termed T₁ and describes the spin-lattice or longitudinal relaxation whereas another process of proton relaxation, termed T₂, specifies the spin-spin or transverse relaxation of the excited protons. The efficiency of the contrast agent is usually expressed as its relaxivity r₁ or r₂, respectively, that is, the ability to shorten the relaxation time per millimole of the contrast agent.

For a first classification, the contrast agents can be divided into two major types. Positive contrast agents act to shorten mainly the relaxation time T₁ and at the same time provide a moderate impact on T₂, thus generating a bright image. Negative contrast agents, on the other hand mainly shorten the transverse relaxation time T₂ and lead to signal reduction, that is a dark image.

Positive (T1-) contrast agents commonly comprise paramagnetic chelates such as Gd-DTPA (Caravan et al, Chemical Reviews 1999; Toth et al, Contrast Agents I 2002 and Na et al, J. Mater. Chem. 2009). Their relaxivity ratio r₂/r₁ commonly is in the range of 1-2. Recently, MnO nanoparticles have also been used although the MnO nanoparticles exhibited low relaxivities (Na et al, Angewandte Chemie-International Edition 2007). The negative (T2-) contrast agents predominantly comprise superparamagnetic iron oxide (SPIO) particles (Na et al, J. Mater. Chem. 2009) that can be roughly classified according to their hydrodynamic sizes. They show high r₂/r₁ ratios of at least 10.

One group of the negative contrast agents are iron oxide particles with hydrodynamic sizes of 40-100 nm that are used to stain cells of the reticulo-endothelial system (RES), i.e. macrophages in the liver or the spleen. Smaller particles of approximately 20 nm size can also be used for MR lymphography. The use of the iron oxide particles as the negative contrast agent arises from the large hydrodynamic diameter of many clinically applied products or controlled clustering (Ai, et al, Advanced Materials 2005; Kim et al, Advanced Materials 2008) of the individual iron oxide particles. Even the single iron oxide particles with a smaller hydrodynamic diameter are preferentially suitable for T₂ weighted MRI due to their strong magnetization at common fields used for MRI which is associated with their superparamagnetism (Lee et al, Nature Medicine 2007; Schellenberger et al, Small 2008). Recently, the impact of surface modification and compartmentalization of superparamagnetic nanoparticles was investigated on negative contrast enhancement and a T₂ contrast agent developed that allows direct imaging of metabolic processes (Tromsdorf et al, Nano Letters 2007; Bruns, et al, Nature Nanotechnology 2009). Other results confirmed the importance of surface chemistry on proton relaxivity (Duan, et al, Journal of Physical Chemistry C 2008).

The use of the iron oxide particles in T₁ weighted imaging is, in most cases, limited due to the large r₂/r₁ ratio, although the impact on the T₁ weighted imaging is significant and often higher compared to paramagnetic chelates. Therefore, only few examples are published so far in which the iron oxide particles are applied as a T₁ contrast agent (Taboada, et al, Langmuir 2007). One example are so-called blood pool contrast agents that are applied to image particular vessel structures in MR angiography (MRA) (Wagner, et al, Investigative Radiology 2002) and provide longer blood half-life compared to the classes described above. The iron oxide based MRA comprises very small ones of the iron oxide particles and are coated with small molecules such as citrate (Taupitz et al, Investigative Radiology 2004). As an advantage over conventional Gd based T₁ contrast agents, the iron oxide particles provide low long term toxicity. The Gd-based contrast agents have been shown recently to be associated with the development of nephrogenic systemic fibrosis in patients with impaired kidney function, a common disease with increasing incidence in the elderly (Penfield et al, Nature Clinical Practice Nephrology 2007). This severe side effect of the Gd-based contrast agents might render these patients wheel-chair dependent and led to new recommendations for the application of these Gd-based contrast agents.

A strategy to form the T₁ contrast agents suitable for MRI out of the iron oxide particles should involve the following aspects. The size of a crystal core must be suitably synthesized for T₁ shortening while the impact on T₂ has to be limited. This is the case for ultrasmall ones (nanoscale) of the iron oxide nanoparticles of core sizes around 5 nm, i.e. in the range 4-6 nm. Second, the organic shell surrounding the core must be designed carefully with respect to stability under physiological conditions as well as a complete prevention of aggregation of the individual nanoparticles which would result in T₂ contrast enhancement again (Josephson et al, Angewandte Chemie-International Edition 2001; Perez et al, Chembiochem 2004, Roch et al, Journal of Magnetism and Magnetic Materials 2005). Third, these nanoparticles should exhibit a low degree of non-specific uptake by phagocytic cells to display a prolonged circulation time.

The European Patent No EP 0 877 630 B1 discloses a superparamagnetic particle based contrast agent, comprising an iron oxide core with a coating of an oxidatively cleaved starch optionally together with a functionalised polyalkylenoxide which serves to prolong blood resistance. The contrast agent of the EP '630 disclosure is characterised at low magnetic fields (0.5 T) suggesting an increasing necessity of low field MR scanners. The contrast agent is not characterized as a positive contrast agent at clinical relevant fields (1.5 T) as both the r₁ coefficient as well as the r₂/r₁ ratio (which actually determines whether the sample acts as a positive contrast agent) decrease with increasing magnetic field strength.

International Patent Application No. WO 2009/051392 (Seoul National University Industry Foundation and Ajou University Industry-Academic Cooperation Foundation) teaches a biocompatible suspension stabiliser for dispersing inorganic nanoparticles into an aqueous solution. The text of the WO '392 application states that the stabiliser can be used as an MRI contrast agent. However, no detailed examples are given of this use. Example 3 of the WO '392 patent application teaches the synthesis of magnetic Fe₃O₄ nanoparticles in an organic solvent and then stabilisation with oleic acid. The magnetic Fe₃O₄ nanoparticles were dispersed into 10 ml of THF. 1 g of phosphorated PEG-derived suspension stabiliser was dissolved in 5 ml of THF and then added to the magnetic Fe₃O₄ nanoparticle dispersion. The THF was evaporated and the resulting adduct heated at 150° C. for one hour under vacuum. Finally 10 ml of water was added to the product.

The teachings of the WO '392 patent do not indicate the size of the core of the magnetic Fe₃O₄ nanoparticles, although FIG. 2 suggests a size of around 10 nm. It is similarly not clear from the description whether the magnetic Fe₃O₄ nanoparticles are used as T1 contrast agents or T2 contrast agents.

A lot of work has been done on the use of PEG as ligand for iron oxide nanocrystals (Kim et al, Journal of the American Chemical Society 2005; Nikolic et al, Angewandte Chemie-International Edition 2006; Thunemann et al, Langmuir 2006; Lattuada et al, Langmuir 2007). However, the coating of the iron oxide nanoparticles with the PEG often results in large hydrodynamic diameters and the formation of at least small amounts of aggregates (Xie et al, Chemistry of Materials 2007; Xie et al, Advanced Materials 2007, which in turn enables these systems to act as T₂ contrast agent.

### SUMMARY OF THE INVENTION

This disclosure teaches T₁ blood pool contrast agent comprising iron oxide nanoparticles of a core size of less than 10 nm, more particularly less than 6 nm and in general between 4 nm and 6 nm, that are coated with poly(ethylene glycol) (PEG) based ligands. The core size and a length of the PEG chain were optimized according to stability, relaxometric properties, cytotoxicity and unspecified cell uptake to enable the iron oxide nanoparticles to be used as a T1 contrast agent for MRI.

A method for the manufacture of monodisperse (less than 10% standard deviation) iron oxide nanoparticles is disclosed with the core sizes of less than 6 nm, in particular between 4 nm and 6 nm, and therefore optimized relaxometric properties. Phosphate functionalized PEG is used for phase transfer to the aqueous solution and the PEG chain length is adjusted in order to prevent aggregation of nanoparticles under physiological conditions and to minimize cytotoxicity and unspecific cell uptake into macrophages.

In one aspect of the invention the core size of the iron oxide nanoparticles is approximately 5 nm and the PEG chain length around 11000

The manufacture of an iron oxide based T₁ contrast agent with a robust PEG coating providing the r₂/r₁ ratio of 2.4 at clinical relevant fields (1.41 T) is disclosed for the PEG coated superparamagnetic iron oxide nanoparticles with the core size between 4 nm and 6 nm. The r₁ relaxivity of 7.3 mM⁻¹s⁻¹ is approximately two times higher than conventional MAGNEVIST® (Gd-DTPA).

It is believed that the T1 contrast agent of this disclosure should provide low long-term toxicity.

### BRIEF DESCRIPTION OF THE FIGURES

FIG. 1 TEM images of the Fe₃O₄ nanoparticles (4 nm core size) coated with oleic acid (a), PEG 550 (b) and PEG 2000 (c).

FIG. 2 GFC analysis of the PEG coated nanoparticles:

FIG. 3 Longitudinal and transverse relaxivity of nanoparticles coated with PEG based ligands of different size

FIG. 4 MTT cytotoxicity assay for J774 macrophages incubated with various PEG coated iron oxide nanoparticles for 24 h.

FIG. 5 Prussian blue staining of J774 macrophages at an iron incubation concentration of 200 μg/ml after 24 h of incubation.

FIG. 6 Schema for the phosphorylation of poly (ethylene glycol) methyl ether

### DETAILED DESCRIPTION OF THE INVENTION

For a complete understanding of the present invention and the advantages thereof, reference is made to the following detailed description in conjunction with the accompanying Figures.

It should be appreciated that the various aspects of the present invention discussed herein are merely illustrative of specific ways to make and use the invention and do not limit the scope of invention when taken into consideration with the claims and the following detailed description and the accompanying Figures.

It should be realised that features from one aspect of the invention can be combined with features from other aspects of the invention.

The use of the term “a”, “an” and “the” as used throughout the description includes plural references unless clearly indicated otherwise.

The invention provides in one aspect a method for the manufacture of monodisperse iron oxide nanoparticles having a core size of less than 10 nm, coated with poly(ethylene glycol), comprising synthesizing the iron oxide nanoparticles and poly(ethylene glycol) based ligands with phosphate anchor groups by mixing poly(ethylene glycol) methyl ether with an excess of POCl₃ and subsequent hydrolysis of the remaining P-Cl groups, coating the ion oxide nanoparticles with the poly(ethylene glycol) by mixing the nanoparticles with a large excess of poly(ethylene glycol), and transferring the iron oxide coated nanoparticles into an aqueous environment

The invention further provides a nanoparticle comprising an iron oxide core with a diameter in the range of 4 to 6 nm coated with poly(ethylene glycol) via a phosphate anchor.

The iron oxide nanoparticles of this disclosure can be used as a T1 contrast agent, a blood pool contrast enhancement (T₁) agent, lymph node imaging agent, targeting imaging agent and hyperthermia agent.

Oleic acid stabilized superparamagnetic Fe₃O₄ (iron oxide or magnetite) nanoparticles (4 and 6 nm mean core diameter) were synthesized as reported previously (Sun et al, Journal of the American Chemical Society 2004; Xie et al, Pure and Applied Chemistry 2006; Sun et al, Journal of the American Chemical Society 2002). The superparamagnetic iron oxide nanoparticles show a narrow size distribution (standard deviation <10%) as confirmed by TEM and the expected fcc spinel structure as well as a typical superparamagnetic behaviour which was demonstrated by magnetization measurements.

In order to synthesize 4 nm sized iron oxide nanoparticles: 2 mmol iron(III) acetylacetonate (iron precursor), 10 mmol 1,2-hexadecanediol (reduction agent), 6 mmol oleic acid (stabilizer), 6 mmol oleyl amine (stabilizer) and 20 phenyl ether (b.p. 260° C.) (solvent) was mixed and heated to 200° C. for 30 min under a flow of nitrogen. Afterwards, the black solution was heated to ˜260° C. under a blanket of nitrogen for 30 min.

After cooling down to room temperature the black solution was separated from the solvent through the addition of ˜50 ml ethanol followed by centrifugation (3260 g, 10 min) and re-dispersion in hexane (5 ml). The nanoparticles were precipitated once more via the addition of ethanol (30 ml) and centrifugation (3260 g, 10 min) and finally dispersed in 5 ml of hexane to form a stable colloidal solution.

To synthesize 6 nm iron oxide nanoparticles benzyl ether (b.p. 300° C.) was used instead of phenyl ether as the solvent. Due to the higher reaction temperature 6 nm sized iron oxide nanoparticles are formed in this example. The iron oxide nanoparticles of larger sizes (8-20 nm) can be synthesized via seed-mediated growth procedures.

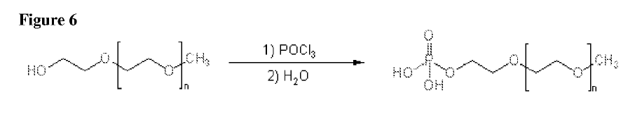

For phase transfer into aqueous medium poly(ethylene glycol) (PEG) based ligands were used which were employed to ligand exchange reactions. To provide robust linkage of the polymers the PEG-based ligands were synthesized with anchor groups which are known to form strong binding to the surface of the iron oxide nanoparticles. For this purpose, phosphates have previously been demonstrated to provide strong binding to the surface of the iron oxide nanoparticles (White et al, Journal of the American Chemical Society 2001; Lalatonne et al, Chemical Communications 2008). The PEG based ligands with various PEG chain lengths were synthesized according to the scheme shown in FIG. 6. To introduce a phosphate group 5 mmol poly(ethylene glycol) methyl ether (mPEG) was used in a reaction with an excess POCl₃ (6 mmol) in tetrahydrofurane followed by subsequent hydrolysis of the remaining two P-Cl groups through the addition of water. Via ³¹P NMR spectroscopy it was confirmed that a phosphate monoester was formed. However, for the short PEG chain (350 g/mol) a second peak of very low intensity in the ³¹P NMR spectrum was observed which probably appears due to a small amount of bi-ester product. The phosphate monoester acts as an anchor group for a robust linkage to the surface of the iron oxide nanoparticles.

For the ligand exchange reaction the iron oxide nanoparticles were directly transferred from tetrahydrofurane (THF) into an aqueous environment after heating to 60° C. with a large excess of the PEG of the desired molar mass. This approach allowed quantitative conversion of the hydrophobic nanocrystals to hydrophilic ones. Furthermore, a minimal length of a PEG chain of ˜500 g/mol was found that is attached to the anchor group is required to circumvent aggregation processes.

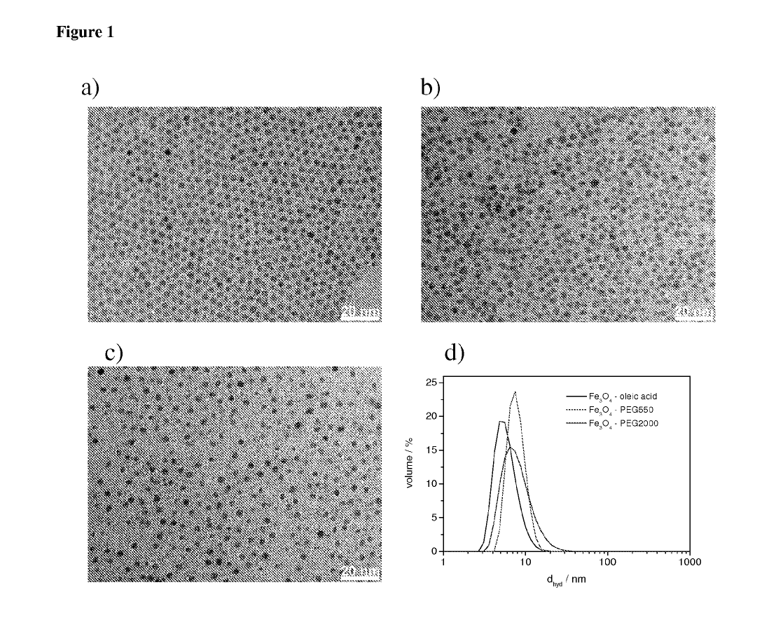

To characterize the ligand exchanged nanoparticles TEM, Dynamic Light Scattering (DLS) and Gel Filtration Chromatography (GFC) was used. By using phosphate-PEG it was possible to synthesize the ion oxide nanoparticles with hydrodynamic diameters of ˜10 nm in water and slightly below as can be seen from FIG. 1d. This seems reasonable for a core size of 4 nm, calculating the hydrodynamic diameter of a PEG 2000 molecule to be 2.8 nm in solution (Sperling et al, Journal of Physical Chemistry C 2007).

In FIG. 1 representative TEM images of the 4 nm sized iron oxide nanoparticles coated with oleic acid (a), PEG 550 (b), and PEG2000 (c) are depicted. As one can observe the iron oxide nanoparticles are evenly distributed after the water is evaporated from the TEM grid with an increasing distance between the iron oxide nanoparticles with increasing polymer chain length. This fact together with the DLS results (FIG. 1d) demonstrates that the iron oxide nanoparticles are homogeneously dispersed in contrast to previous results where aggregates or worm-like structures were obtained. This may be attributed to the strong bond of the phosphate group to the iron oxide nanoparticle surface as well as the complete absence of any hydrophobic part within the ligand structure.

All dispersions of the iron oxide nanoparticles show high stability under various pH treatments, and under ionic strength up to 2 M of NaCl and various buffer systems without any change in hydrodynamic diameter and therefore without any aggregation as confirmed by DLS measurements in good agreement with other results (Wu et al, Angewandte Chemie-International Edition 2008).

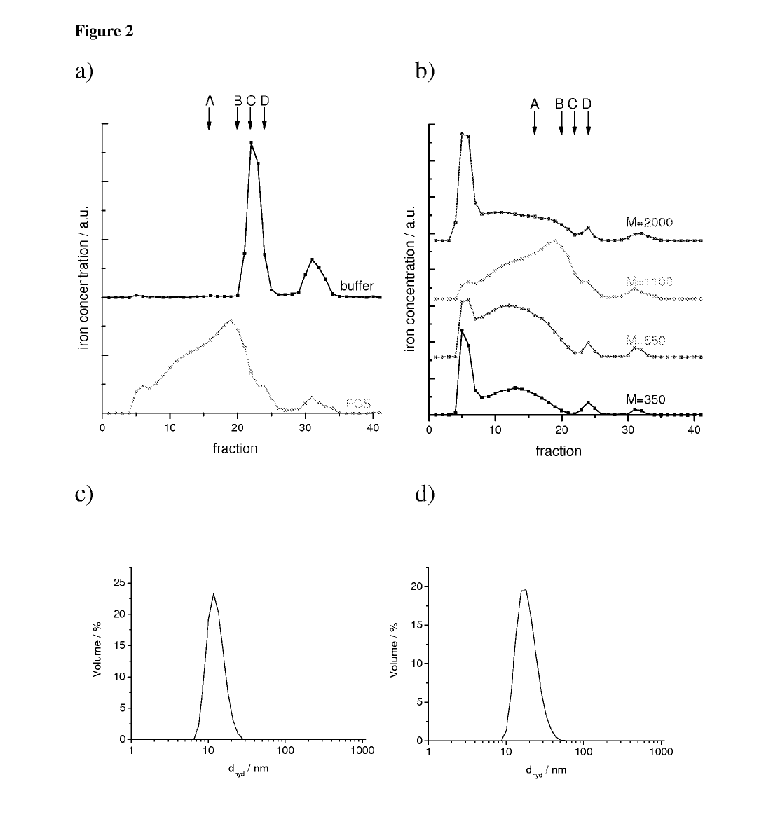

The stability of the prepared iron oxide nanoparticles was investigated under physiological conditions using GFC as this method is very sensitive to small changes in the hydrodynamic diameter. This is useful for a T₁ blood pool contrast agent because an aggregation would provide a strong impact on T₂. Therefore, the iron oxide nanoparticles were incubated in fetal calf serum (FCS) for 2 h at 37° C. and the obtained GFC curve was compared to a corresponding sample that was incubated in a Tris/NaCl-buffer under the same conditions (FIG. 2a).

The results most likely demonstrate the adsorption of the plasma proteins to the iron oxide nanoparticle surface and, as a consequence, a slight increase in hydrodynamic diameter. The PEG-based ligands were tested with other anchor groups such as carboxylic acid but similar results were obtained. This behaviour is in contrast to other systems like CdSe/ZnS where the adsorption could be completely prevented (Choi et al, Nature Biotechnology 2007). However, significant differences were observed with respect to the polymer chain length that is attached to the phosphate anchor group (FIG. 2b). For the smallest polymer chain (M=350 g/mol) the strongest increase in the hydrodynamic diameter due to an insufficient stabilization of the nanoparticles in solution was observed. A substantial part of the iron oxide nanoparticles in the early GFC fractions (F5-6) was found. The use of polymers with higher molar masses (PEG 550, PEG 1100) resulted in a smaller hydrodynamic diameter that is a higher stability against aggregation processes that might be induced by plasma proteins although their adsorption could not be completely prevented. In addition, DLS measurements of particular GFC fractions (F12, F18) verified, that the hydrodynamic diameter increased slightly (FIG. 2c,d). Therefore, it can be concluded that the iron oxide nanoparticles have a final hydrodynamic diameter of 10-15 nm in serum.

To characterize the relaxometric properties MR measurements were performed at 1.41 T (60 MHz) in order to investigate the impact of the coating of the iron oxide nanoparticles with the various ligands on the ability to shorten the longitudinal relaxation time T₁ and the transverse relaxation time T₂ and thus whether the sample is suitable as a T₁ contrast agent.

The influence of the core size of the nanoparticles, the size of the ligand and induced aggregation on the T₁ and T₂ relaxation processes were investigated. A possible dependence of the contrast enhancement on the nature of the stabilizing surfactants has been reported (Duan et al, Journal of Physical Chemistry C 2008). Moreover, the impact of the slight increase in the hydrodynamic diameter in the serum on the relaxation processes was investigated besides determination of the longitudinal (r₁) and transverse (r₂) relaxivities of the various samples by measuring the characteristic relaxation times of a concentration series and plotting the inverse relaxation time that is the relaxation rate against the ionic iron concentration. The slope of the as determined straight line is defined as the relaxivity and represents the efficiency of the contrast agent. The relaxivities of four individual samples were determined which differ in the length of the used PEG chain. The same PEG molar masses were used as described above for the serum stability tests. The relaxivity of a sample comprising the 6 nm sized iron oxide nanoparticles was also determined. Besides the absolute relaxivities of a contrast agent another useful factor is the value of r₂/r₁ as it ascertains whether the considered sample acts as a T₁ or a T₂ contrast agent. For a T₁ contrast agent r₂/r₁ should be as small as possible.

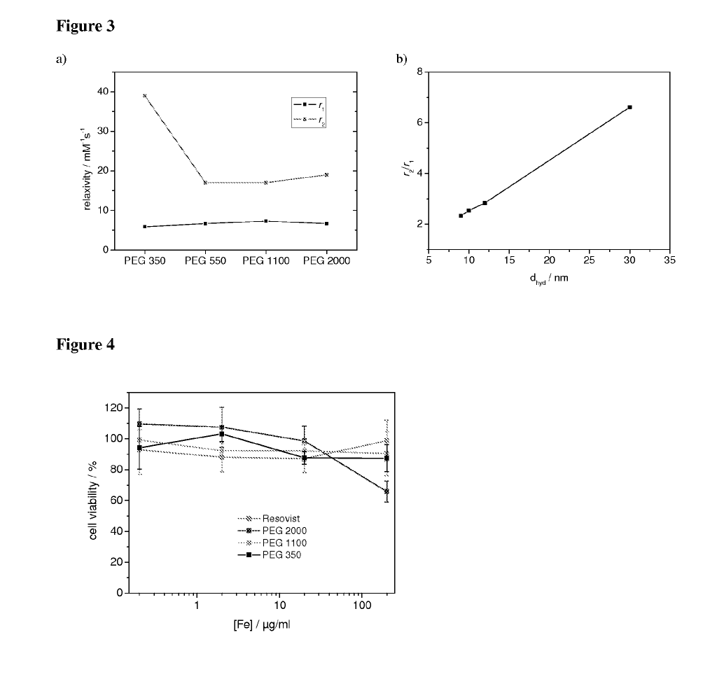

First of all it can be seen from FIG. 3a that all samples have comparable r₁ values with respect to the size of the PEG chain whereas r₂ strongly varies. This discrepancy in r₂ is obviously due to aggregation effects which are known to be responsible for significant shortening of the transverse relaxation time (Perez et al, Nature Biotechnology 2002). Therefore, these results confirm the fact that a minimal PEG chain molecular mass of 550 g/mol is necessary in this case to synthesize individually dispersed nanoparticles in aqueous solution without any tendency to aggregation.

However, the smallest hydrodynamic diameter below 10 nm was obtained using PEG 1100. In this case a longitudinal relaxivity r₁=7.3 mM⁻¹s⁻¹ and a r₂/r₁ ratio of 2.4 at 1.41 T was measured which makes this sample a candidate as use as a T1 contrast agent for positive image generation at clinical relevant magnetic fields. For comparison, the relaxivities of the typical T₂ contrast agent RESOVIST® are r₁=11 mM⁻¹s⁻¹ and r₂=130 mM⁻¹s⁻¹ (1.41 T). Hence, the optimized contrast agent according to the invention has a comparable r₁ value whereas r₂ could be strongly limited. Moreover, MAGNEVIST® as a typical Gd based T₁ contrast agent provides a r₁ relaxivity of 3.6 mM⁻¹s⁻¹ at 1.41 T which is significantly lower compared to the value of the T1 contrast agent as described in this disclosure. Furthermore the r₂/r₁ ratio is comparable to other ones of the iron oxide contrast agents that are under investigation for MR angiography (Taupitz et al, Investigative Radiology 2004) and is surprisingly the smallest value for the PEG coated iron oxide nanoparticles at all. The use of larger PEG-based ligands yielded samples with a higher r₂/r₁ ratio thus demonstrating an increasing tendency to aggregation. This fact might be due to a less dense occupancy of the PEG chains on the iron oxide nanoparticle surface. FIG. 3b points out that the hydrodynamic diameter strongly correlates with r₂/r₁.

In order to check whether the iron oxide nanoparticles keep their relaxometric properties under physiological conditions the relaxation times in FCS were determined. The adsorption of plasma proteins to the iron oxide nanoparticle surface did not change the spin-lattice and spin-spin relaxation times over a period of 24 h, a fact that points once more out that the iron oxide nanoparticles remain individually dispersed. The relaxation times of the same GFC fractions that have been investigated by DLS (FIG. 2c,d) of the PEG 1100 sample were incubated in FCS for 2 h. A spin-lattice relaxation time T₁=911 ms and a spin-spin relaxation time T₂=369 ms was measured. The T₁/T₂ ratio was 2.5 and thus close to the r₂/r₁ ratio that was determined in water (2.4). Therefore, one can conclude that the iron oxide nanoparticles fully keep their magnetic and relaxometric properties although plasma proteins adsorb and thus slightly increase their hydrodynamic diameter.

As the use of PEG 1100 lead to most satisfactory results in terms of stability and relaxivity this polymer was used in order to investigate the impact of a slight increase in the iron oxide nanoparticle core size from 4 to 6 nm. An increase of both longitudinal and transverse relaxivity. r₁ with an increase to 13 mM⁻¹s⁻¹ was observed while r₂ increased to 42 mM⁻¹s⁻¹ at 1.41 T resulting in a r₂/r₁ ratio of 3.2. Interestingly, the small size difference of 2 nm resulted in a 2.5-fold increase of the transverse relaxivity and a 2-fold increased in the longitudinal relaxivity, while the hydrodynamic diameter remained at approximately 10 nm.

In contrast, the use of 4 nm sized iron oxide nanoparticles and the short PEG 350 chain resulted in a significant increase in the hydrodynamic diameter to 30 nm due to clustering of individual ones of the iron oxide nanoparticles. At the same time, r₂ increases to 39 mM⁻¹s⁻¹ whereas r₁ even decreases to 5.9 mM⁻¹s⁻¹. This demonstrates that although a clustering in solution leads to an increase of r₂, r₁ decreases at the same time. This might be due to the smaller surface of the cluster compared to homogeneously dispersed iron oxide nanoparticles. A simple increase in core size results on the other hand in an increase in r₂ and r₁. This is probably a consequence of the higher saturation magnetization of the larger nanocrystals. However, r₂/r₁ also increases with the increasing core size. Therefore, the results suggest that there is indeed a size limit for the superparamagnetic core of approximately 5 nm if the iron oxide nanoparticles should act as T₁ contrast agent and any aggregation processes should be substantially prevented.

As a relevant biological system for investigations of cytotoxic effects and cell uptake J774 mouse macrophage cells were used. The macrophages cells are phagocytes that belong to the reticulo-endothelial system (RES) and are predominantly localized in the liver, spleen and bone marrow. These macrophage cells are, in particular, interesting because each nanoparticle contrast agent applied would experience phagocytosis after certain time of circulation if there is no specify through bio-functionalisation in terms of molecular or cellular imaging or the nanoparticles exhibit hydrodynamic diameters below ˜6 nm thus allowing renal clearance. For a blood pool contrast agent circulation times should be long because the contrast agent should provide low levels of phagocytosis.

To estimate a biologically reasonable incubation concentration for the cytotoxicity investigations the following assumptions were made: Based on typical injection doses in a mouse experiment (0.2 ml injection volume, 2 mg Fe/ml concentration, 2 ml blood volume) an incubation concentration of 200 μg/ml is referred to as biological relevant concentration in the further discussion. A standard MTT assay with various incubation concentrations (0.2-200 μg Fe/ml) was performed and several PEG coated iron oxide nanoparticle contrast agents as well as RESOVIST® a clinically applied contrast agent based on iron oxide (FIG. 4) were tested. The PEGylated iron oxide nanoparticles provide low cytoxicity and are comparable to RESOVIST®, the clinical standard, in this regard. This is in good agreement with previous investigations on the iron oxide nanoparticles coated with PEG (Gupta et al, IEEE Transactions on Nanobioscience 2004). However, the nanoparticles coated with PEG 2000 lead to a relevant reduction of the cell viability at the highest concentration.

In further experiments phagocytosis of the PEGylated iron oxide nanoparticles was investigated using the J774 macrophages which are murine macrophages. It would be useful to know for a potential application as blood pool contrast agent whether the PEG coating results in a significant decrease of unspecific uptake into cells of the RES.

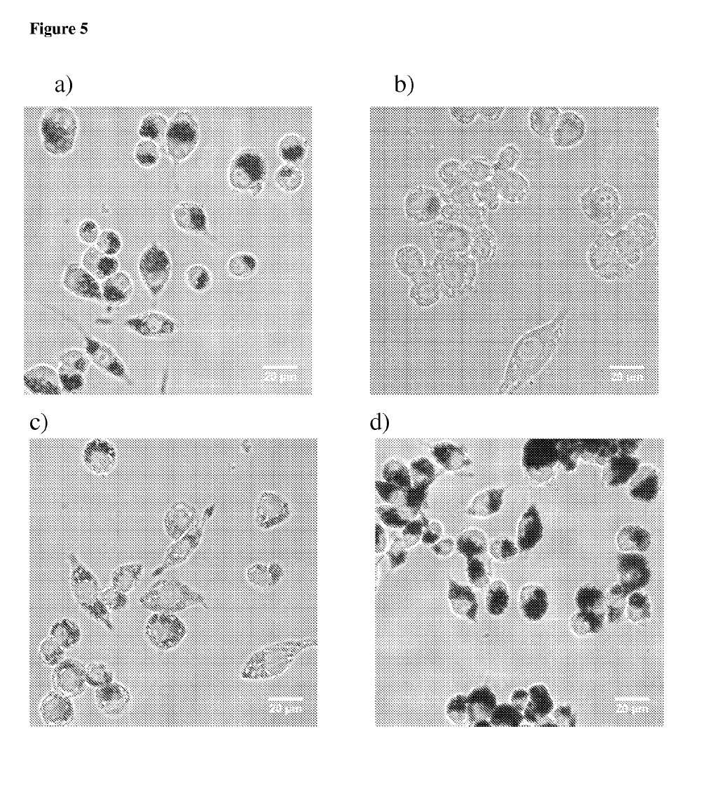

Representative images of the J774 macrophage cells which are stained with Prussian blue are shown in FIG. 5. First, it can be noticed that all investigated PEG coatings lead to a reduction of cell uptake compared to clinically used RESOVIST®. However we also observed significant differences with respect to the length of the used PEG chain. The lowest uptake level was found for PEG 1100 coated iron oxide nanoparticles. However, for the shorter (PEG 350) and longer (PEG 2000) chains higher degrees of contrast agent uptake could be observed. The results are in good agreement with those obtained in the stability investigations (described above), where the PEG 1100 coated iron oxide nanoparticles lead to lowest increase in hydrodynamic diameter meaning the highest resistance against the adsorption of plasma proteins.

A T₁ blood pool contrast agent based on the very small iron oxide nanoparticles (4 nm core size) was produced. Although a coating with PEG 1100 could not completely avoid the adsorption of serum proteins, the increase in the hydrodynamic diameter is small and the iron oxide nanoparticles surprisingly fully keep their relaxometric properties under physiological conditions. The final hydrodynamic diameter in serum is about 10-15 nm. The smallest possible r₂/r₁ ratio was 2.4 at clinical field strength (1.41 T) and is thus comparable or even lower than other iron oxide based systems with citrate as charge stabilizing ligand which are under investigation for T₁ weighted MRI. In addition, the r₁ relaxivity is comparable to clinically used iron oxide based RESOVIST® while r₂ is a factor of seven lower and could therefore be strongly limited a fact that is required for T₁ weighted MRI. On the other hand, r₁ is approximately two times higher than that of MAGNECIST®. The iron oxide nanoparticles described have the smallest r₂/r₁ ratio for PEGylated iron oxide nanoparticles reported so far.

The experimental results suggest that for the manufacture of the T₁ contrast agent based on the iron oxide nanoparticles a core size of approximately 5 nm should be used. If the iron oxide nanoparticle core size is too small the r₁ values are relatively low. An increase of the core size on the other hand leads to an increase in both r₁ and r₂/r₁. Thus, core sizes larger than 6 nm are excluded in terms of an application as the T₁ contrast agent. As an advantage, the iron oxide contrast agent disclosed provides low cytoxicity as preliminary in vitro tests demonstrate and furthermore provide low levels of unspecific uptake into cells of the RES. Under all parameters tested, the PEG 1100 coated iron oxide nanoparticles (core size 4 nm) present an optimum providing highest stability together with suitable relaxometric properties, lowest cytotoxicity and lowest uptake into macrophages.

The iron oxide nanoparticles of the present disclosure require only one modification of the iron oxide cores. It will be noted that the iron oxide nanoparticles of EP 0 877 630 B1 are subject to an at least two step modification process where the starch must be chemically cleaved in a first step using oxidants to release the iron oxide nanoparticles, followed by a second surface modification that might optionally be carried out in order to introduce other ligands to the particle surface. According to the disclosure of EP 0 877 630 B1, the combination of cleaved starch and methoxy-PEG-phosphate leads to the longest blood lifetimes.

Using the method of the present invention it is possible to precisely control the size of the crystalline inorganic core. The size distribution of the crystalline inorganic core is very narrow (standard deviation <10%). This is advantageous since
a. the saturation magnetisation M_{S} is strongly dependent on the size of the core. As a result, the relaxometric properties (i.e. the relaxivity coefficients r₁ and r₂) are also dependent on the size of the core.
b. a broad size distribution which usually occurs using aqueous syntheses (as in EP 0 877 630 B1) causes only a small part of the whole sample to have the desired properties
c. crystallinity and composition are highly controllable

It will be noticed that the iron oxide nanoparticles obtained by the method of the present disclosure have lower magnetization values, even at high magnetic fields. As a consequence, the transverse relaxivity coefficients r₂ are also significantly reduced. This will minimize negative side effects, such as susceptibility artefacts.

Through an exact size control, e.g. 4 nm vs. 6 nm core sized nanoparticles, it is possible to fine tune the magnetic and relaxometric properties with respect to a desired application (e.g. positive MR contrast or local hyperthermia).

It will be appreciated that the PEG-Ligands with the phosphate anchor groups are not only suitable for use with magnetite, but also with other metal oxides such as manganese II oxide.

The synthesis process of the nanoparticles of the current disclosure has two steps. The first step is the synthesis of the iron oxide nanoparticle core and the second step is the introduction of the methoxy-PEG-phosphate. The introduction of a dense methoxy-PEG-phosphate coating as described in the EP 0 877 630 B1 requires the oxidative cleavage of the native starch followed by an intermediate charge stabilisation which might be obtained through the addition of an electrostatic stabilizer like sodium diphosphate. The necessity of these numerous steps compromises the reproducibility of the contrast agent.

The high stability of the iron oxide nanoparticles according to the disclosure was demonstrated using the example of one particular coating molecule (methoxy-PEG1100-phosphate) with very high reproducibilty. This molecule provides a dense coating resulting in the smallest hydrodynamic diameter (d_{hyd}<10 nm), highest resistance against the adsorption of serum proteins, optimized relaxometric properties lowest cytotoxicity and lowest levels of unspecific phagocytosis. All these facts lead to prolonged blood lifetimes of the iron oxide nanoparticle.

### DETAILED DESCRIPTION OF THE FIGURES

FIG. 1 shows TEM images of the Fe₃O₄ nanoparticles (4 nm core size) coated with oleic acid (a), PEG 550 (b) and PEG 2000 (c). An increasing distance between the nanoparticles with increasing polymer chain length is observable. Together with the DLS results (d) it can be concluded that no aggregation occurs during the ligand exchange procedure. A hydrodynamic diameter of 10 nm seems reasonable assuming a simple addition of the core size and the calculated hydrodynamic diameter of a PEG 2000 molecule (d_{eff,PEG}=0.03824 M_{W}^{0.559} according to Sperling et al, Journal of Physical Chemistry C 2007).

FIG. 2 shows a GFC analysis of the PEG coated nanoparticles: a) GFC curves of PEG1100 coated nanoparticles incubated for 2 h in buffer (black) and FCS (red). b) Comparison of various PEG chain lengths in terms of stability in FCS (2 h, 37° C.). The highest stability against the adsorption of plasma proteins was observed for PEG 1100 coated nanoparticles. MW markers A (Thyroglubulin, 669 kDa), B (Apoferritin, 443 kDa), C (Amylase, 200 kDa), D (Albumin, 66 kDa) are shown by arrows. DLS measurements of the GFC fractions F18 (c) and F12 (d) show a slight increase in the hydrodynamic diameter although no aggregation takes place.

FIG. 3 shows a longitudinal and transverse relaxivity of the iron oxide nanoparticles coated with PEG based ligands of different size (a). The value of r₂/r₁ strongly correlates with the hydrodynamic size of the iron oxide nanoparticles in solution (b): For PEG 350 coated iron oxide nanoparticles the tendency to aggregation (d_{hyd}=30 nm) results in significantly higher r₂/r₁ ratio.

FIG. 4 shows a MTT cytotoxicity assay for J774 macrophages incubated with various PEG coated iron oxide nanoparticles for 24 h. As a reference RESOVIST® was used. Even at high iron concentrations (200 μg/ml) PEG 1100 and PEG 350 coated nanoparticles remain non-toxic. PEG 2000 leads to reduced cell viability at this concentration level.

FIG. 5 depicts a Prussian blue staining of J774 macrophages at an iron incubation concentration of 200 μg/ml after 24 h of incubation. Different levels of intracellular contrast agent uptake are clearly observable for the various samples: PEG 350 (a), PEG 1100 (b), PEG 2000 (c) and clinical standard Resovist (d). PEG 1100 coated iron oxide nanoparticles show the lowest degree of unspecific uptake due to the dense PEG coating in good agreement with the stability tests shown above.

FIG. 6 depicts a schema for the phosphorylation of the mPEG molecules

Having thus described the present invention in detail, it is to be understood that the foregoing detailed description of the invention is not intended to limit the scope of the invention thereof. The person skilled in the art will recognise that the invention can be practiced with modification within the scope of the attached claims. At least, it should be noted that the invention is not limited to the detailed description of the invention and/or of the examples of the invention. What is desired to be protected by letters patent is set forth in the following claims.

### REFERENCES

[1] A. Bjornerud, L. Johansson, NMR in Biomedicine 2004, 17, 465.

[2] J. W. M. Bulte, D. L. Kraitchman, NMR in Biomedicine 2004, 17, 484.

[3] Y. X. J. Wang, S. M. Hussain, G. P. Krestin, European Radiology 2001, 11, 2319.

[4] P. Caravan, J. J. Ellison, T. J. McMurry, R. B. Lauffer, Chemical Reviews 1999, 99, 2293.

[5] E. Toth, L. Helm, A. E. Merbach, Contrast Agents 12002, 221, 61.

[6] H. B. Na, J. H. Lee, K. J. An, Y. I. Park, M. Park, I. S. Lee, D. H. Nam, S. T. Kim, S. H. Kim, S. W. Kim, K. H. Lim, K. S. Kim, S. O. Kim, T. Hyeon, Angewandte Chemie-International Edition 2007, 46, 5397.

[7] H. Ai, C. Flask, B. Weinberg, X. Shuai, M. D. Pagel, D. Farrell, J. Duerk, J. M. Gao, Advanced Materials 2005, 17, 1949.

[8] J. Kim, J. E. Lee, S. H. Lee, J. H. Yu, J. H. Lee, T. G. Park, T. Hyeon, Advanced Materials 2008, 20, 478.

[9] J. H. Lee, Y. M. Huh, Y. Jun, J. Seo, J. Jang, H. T. Song, S. Kim, E. J. Cho, H. G. Yoon, J. S. Suh, J. Cheon, Nature Medicine 2007, 13, 95.

[10] E. Schellenberger, J. Schnorr, C. Reutelingsperger, L. Ungethum, W. Meyer. M. Taupitz, B. Hamm, Small 2008, 4, 225.

[11] U. I. Tromsdorf, N. C. Bigall, M. G. Kaul, O. T. Bruns, M. S. Nikolic, B. Mollwitz, R. A. Sperling, R. Reimer, H. Hohenberg, W. J. Parak, S. Forster, U. Beisiegel, G. Adam, H. Weller, Nano Letters 2007, 7, 2422.

[12] O. T. Bruns, H. Ittrich, K. Peldschus, M. G. Kaul, U. I. Tromsdorf, J. Lauterwasser, M. S. Nikolic, B. Mollwitz, M. Merkel, N. C. Bigall, S. Sapra, R. Reimer, H. Hohenberg, H. Weller, A. Eychmüller, G. Adam, U. Beisiegel, J. Heeren, Nature Nanotechnology 2009.

[13] H. W. Duan, M. Kuang, X. X. Wang, Y. A. Wang, H. Mao, S. M. Nie, Journal of Physical Chemistry C 2008, 112, 8127.

[14] E. Taboada, E. Rodriguez, A. Roig, J. Oro, A. Roch, R. N. Muller, Langmuir 2007, 23, 4583.

[15] S. Wagner, J. Schnorr, H. Pilgrimm, B. Hamm, M. Taupitz, Investigative Radiology 2002, 37, 167.

[16] M. Taupitz, S. Wagner, J. Schnorr, Investigative Radiology 2004, 39, 625.

[17] J. G. Penfield, R. F. Reilly, Nature Clinical Practice Nephrology 2007, 3, 654.

[18] L. Josephson, J. M. Perez, R. Weissleder, Angewandte Chemie-International Edition 2001, 40, 3204.

[19] J. M. Perez, L. Josephson, R. Weissleder, Chembiochem 2004, 5, 261.

[20] A. Roch, Y. Gossuin, R. N. Muller, P. Gillis, Journal of Magnetism and Magnetic Materials 2005, 293, 532.

[21] S. W. Kim, S. Kim, J. B. Tracy, A. Jasanoff, M. G. Bawendi, Journal of the American Chemical Society 2005, 127, 4556.

[22] M. S. Nikolic, M. Krack, V. Aleksandrovic, A. Kornowski, S. Forster, H. Weller, Angewandte Chemie-International Edition 2006, 45, 6577.

[23] A. F. Thunemann, D. Schutt, L. Kaufner, U. Pison, H. Mohwald, Langmuir 2006, 22, 2351.

[24] M. Lattuada, T. A. Hatton, Langmuir 2007, 23, 2158.

[25] J. Xie, C. J. Xu, Z. H. Xu, Y. L. Hou, K. L. L. Young, S. X. Wang, N. Pourmand, S. H. Sun, Chemistry of Materials 2007, 19, 1202.

[26] J. Xie, C. Xu, N. Kohler, Y. Hou, S. Sun, Advanced Materials 2007, 19, 3163.

[27] S. Z. Sun, Hao; Robinson, David B.; Raoux, Simone; Rice, Philip M.; Wang, Shan X.; Li, Guanxiong, Journal of the American Chemical Society 2004, 126, 273.

[28] J. Xie, S. Peng, N. Brower. N. Pourmand, S. X. Wang, S. H. Sun, Pure and Applied Chemistry 2006, 78, 1003.

[29] S. H. Sun, H. Zeng, Journal of the American Chemical Society 2002, 124, 8204.

[30] M. A. White, J. A. Johnson, J. T. Koberstein, N. J. Turro, Journal of the American Chemical Society 2007, 129, 4504.

[31] Y. Lalatonne, C. Paris, J. M. Serfaty, P. Weinmann, M. Lecouvey, L. Motte, Chemical Communications 2008, 2553.

[32] R. A. Sperling, T. Liedl, S. Duhr, S. Kudera, M. Zanella, C. A. J. Lin, W. H. Chang, D. Braun, W. J. Parak, Journal of Physical Chemistry C 2007, 111, 11552.

[33] H. Wu, H. Zhu, J. Zhuang, S. Yang, C. Liu, Y. C. Cao, Angewandte Chemie-International Edition 2008, 47, 3730.

[34] H. S. Choi, W. Liu, P. Misra, E. Tanaka, J. P. Zimmer, B. I. Ipe, M. G. Bawendi, J. V. Frangioni, Nature Biotechnology 2007, 25, 1165.

[35] J. M. Perez, L. Josephson, T. O'Loughlin, D. Hogemann, R. Weissleder, Nature Biotechnology 2002, 20, 816.

[36] A. K. Gupta, S. Wells, IEEE Transactions on Nanobioscience 2004, 3, 66.

[37] H. B. Na and T. Hyeon, J. Mater Chem, 2009, 19, 6267-6273.

1. A method for the manufacture of monodisperse iron oxide nanoparticles having a core size of less than 10 nm and being coated with poly(ethylene glycol) comprising the steps of:
a. synthesizing
i. the iron oxide nanoparticles and poly(ethylene glycol) based ligands with phosphate anchor groups by mixing poly(ethylene glycol) methyl ether with an excess of POCh and subsequent hydrolysis of the remaining two P-CI groups; and
ii. coating the iron oxide nanoparticles with the poly(ethylene glycol) by mixing the nanoparticles with a large excess of poly(ethylene glycol); and
c. transferring the coated iron oxide nanoparticles into an aqueous environment.

2. The method of claim 1, wherein the iron oxide nanoparticles have core sizes between 4 to 6 nm.

3. The method of claim 2, using a mixture of iron oxide nanoparticles with a defined ratio of nanoparticles with a core size between 4 and 6 nm.

4. The method according to claim 1, wherein for synthesizing the iron oxide nanoparticles an iron precursor, a reduction agent, stabilizer and a first solvent are mixed to form a black solution and heated followed by separating the black solution from the first solvent by a precipitation step by adding an alcohol and centrifugation with subsequent re-dispersion in a solvent and repeating the precipitation step before final dispersing in a second solvent.

5. The method according to claim 4, wherein phenyl ether is used as the first solvent in the mixture for synthesis of the iron oxide cores with 4 nm diameter and benzyl ether for the synthesis of the iron oxide cores with 6 nm diameter.

6. The method according to claim 1, wherein the iron oxide nanoparticles are oleic acids stabilized superparamagnetic nanoparticles.

7. The method according to claim 1, wherein the coating of the iron oxide nanoparticles comprises heating to 60° C.

8. The method according to claim 1, wherein the coated iron oxide nanoparticles are transferred from tetrahydrofurane into an aqueous environment

9. The method according to claim 1, wherein the poly(ethylene glycol) chain length is adjusted by using a poly(ethylene glycol) with a molecular mass in the range of 400 to 2000 g/mol, preferably with a minimum chain length of about 550 g/mol, and most preferably with a chain length of 1100 g/mol.

10. The method according to claim 1 wherein the iron oxide is magnetite.

11. A nanoparticle comprising a iron oxide core with a diameter in the range of 4 to 6 nm coated with a poly(ethylene glycol) polymer via a phosphate anchor.

12. The nanoparticle of claim 11 having a hydrodynamic diameter in the range of 10 to 15 nm.

13. The nanoparticle of claim 11, wherein a chain length of the poly(ethylene glycol) polymer is adjusted by using a poly(ethylene glycol) polymer with a minimum molecular mass of about 550 g/mol, and preferably 1100 g/mol.

14. The nanoparticle according to claim 11, wherein the longitudinal relaxivity r₁ is in the range from 7.3 to 13 mM⁻¹s⁻¹ and the r₂/r₁ ratio is in the range from 2.4 to 3.2 at 1.41 T.

15. A composition comprising a nanoparticle according to claim 11.

16. The composition of claim 15 further comprising physiologically tolerable carrier or stabilizer.

17. The use of a nanoparticle manufactured according to the method of claim 1 as one of a contrast agent, a blood pool contrast enhancement (T1) agent, lymph node imaging agent, targeting imaging agent and hyperthermia agent.

18. The use of a nanoparticle resulting from the method of claim 1 for the manufacture of a contrast agent or contrast agent composition.

19. The use of a nanoparticle resulting from the method of claim 1 in the prophylaxis, diagnosis, therapy, follow-up and/or aftercare of a therapy.

20. The use of a nanoparticle resulting from the method of claim 1 in imaging methods.
